(19) United States
(12) Patent Application Publication
(10) Pub. No.: US 2010/0139366 A1
(43) Pub. Date: Jun. 10, 2010

(54) LAUNDRY TREATMENT APPLIANCE WITH GAS SENSOR AND METHOD FOR TREATING LAUNDRY

(75) Inventor: Uwe-Jens Krausch, Brieselang (DE)

Correspondence Address: BSH HOME APPLIANCES CORPORATION;INTELLECTUAL PROPERTY DEPARTMENT, 100 BOSCH BOULEVARD, NEW BERN, NC, 28562, US

(73) Assignee: BSH BOSCH UND SIEMENS HAUSGERÄTE GMBH, Munich (DE)

(21) Appl. No.: 12/628,240

(22) Filed: Dec. 1, 2009

(30) Foreign Application Priority Data
Dec. 10, 2008 (DE) 10 2008 054 462.0

(51) Int. Cl. G01N 33/00; D06F 35/00

(52) U.S. Cl. 73/23.34; 68/13 R

(57) ABSTRACT

The invention relates to a laundry treatment appliance laundry treatment appliance including a container to hold laundry items, and an odor sensor with at least two gas sensors that are different in respect of their reaction to different chemical characteristics of a predetermined gas or gas mixture.

### BACKGROUND OF THE INVENTION

The invention relates to a laundry treatment appliance with a gas sensor and a method for treating laundry using the laundry treatment appliance.

Methods and apparatuses have already been developed for laundry treatment appliances, in particular dryers, washing machines and washer/dryers to neutralize unpleasant odors in laundry items and/or to treat laundry items with fragrances or similar.

For example EP 1 327 019 B1 describes an apparatus for treating fabrics in a drum-type dryer. Cyclodextrins are specified as agents for neutralizing odors and as perfume enhancers. These and numerous other substances are used according to EP 1 327 019 B1 for fabric conditioning and are enclosed to this end in a membrane, which prevents the egress of liquids but not the egress of gases. During the operation of a dryer the action of heat causes liquids escaping from the membrane jacket to evaporate and be distributed in the dryer and the laundry therein.

WO 2007/036470 A1 discloses a method and a facility, in particular a tumble dryer, in which in addition to the laundry items to be treated a further laundry item is used as a filter, to absorb the vapors and substances coming out of the other laundry items.

EP 1 431 443 A1 discloses a tumble dryer, which is fitted with an ultrasonic atomizer, which is to be used to introduce substances, for example fragrances, into the drum.

In EP 0 676 497 B1 a method for spraying perfumed oil onto laundry by means of a pressurized spray nozzle is described, the fragrance being sprayed onto already dried laundry, while the drum rotates continuously to distribute the fragrance evenly.

WO 2007/087937 A1 discloses a washing machine with a facility for deodorizing clothes. To this end a component with a spray nozzle is provided in the seal of the laundry drum and is able to emit an active agent (e.g. cyclodextrins) in the form of a spray mist into the interior of the drum. A facility for fragrancing clothes in a tumble dryer is known from WO 2004/059070 A1.

These known methods assume that the presence of odors, in particular of chemical substances causing unpleasant odors, so-called odorous substances, has already been determined or that a deodorizing method or a method providing a pleasant odor is implemented regardless of the odor status of the laundry. Prior detection of odorous substances does not take place in the laundry treatment appliance.

The use of sensors to determine operating conditions in a laundry treatment appliance is also known. Thus for example the use of INFRARED sensors for identifying textile types, fill level and quantity of water in the drum of washing machines and tumble dryers is known.

U.S. Pat. No. 5,396,715 describes a microwave tumble dryer and a fire protection method. An INFRARED sensor is used here to register the temperature within the tumble dryer and when a predetermined value is reached, which indicates the combustion of laundry, operation is interrupted. Drums with INFRARED sensor apparatuses for measuring the temperature in tumble dryers are also known from JP-A-06-126099, JP-A-07-178293 and JP-A-05-200194. The use of INFRARED turbidity sensors is described in JP-A-06-039189.

US 2002/000495 A1 relates to systems for controlling drying cycles in a dryer, which contains the vapor of a lipophile liquid, with a gas sensor being used to determine its concentration. Numerous sensors are described, which can be made of different materials and in which various measuring principles can be realized. Sorption sensors are thus described, with which sorption can be detected for example based on the electrical resistance of a measuring surface as well as the use of conductive polymers, the conductivity of which changes when certain gases are adsorbed.

EP 1 602 766 A2 relates to a deodorizing unit for a washing machine and a check method. The odor of the laundry in the washing machine is preferably determined using an odor sensor. The odor sensor uses an electronic nose, which detects gas molecules. Such an electronic nose can be a sensor of the array type, comprising a plurality of gas sensors, or alternatively a micro-type sensor.

DE 37 22 983 A1 discloses a sensor-led and microprocessor-controlled detection and control system for eliminating odors during combustion, coking, gasification and in biological conversion processes. A sensor system is calibrated to a limit value based on olfactometric odor measurement. Continuously operating semiconductor sensors, preferably stannic oxide sensors, are deployed as gas sensors.

### BRIEF SUMMARY OF THE INVENTION

One object of the invention is to provide a laundry treatment appliance and a method for determining the presence of an odorous substance in a laundry treatment appliance, with which prompt identification of critical states, e.g. a fir, in a laundry treatment appliance is preferably possible.

An exemplary embodiment of the invention is a laundry treatment appliance with a container to hold laundry items and a gas sensor, the laundry treatment apparatus having an odor sensor comprising a number of gas sensors, with at least two gas sensors being different in respect of their reaction to different chemical characteristics of a predetermined gas or gas mixture.

A gas sensor within the meaning of the invention is a sensor for a substance, which is present at least partially in the gaseous state at room temperature. “Gas” in the context of the invention therefore means not only in the narrower sense a substance which is present in the gaseous state at room temperature but also those components of a more or less volatile substance, which are present in each instance as an effective component in the gaseous phase at room temperature due to a finite vapor pressure. Since odor is a complex phenomenon, in an odor sensor in the context of the invention at least two gas sensors are specified as different, in other words for different substances.

In the inventive odor sensor at least two gas sensors, preferably 3 to 50 and particularly preferably 5 to 40 gas sensors are deployed.

The gas sensors of the odor sensor preferably comprise an electrically conductive material, the electrical resistance of which changes on contact with a volatile chemical compound.

In one preferred embodiment the electrically conductive material is a semiconducting metal oxide or an electrically conducting polymer. The semiconducting metal oxide here comprises a stannic oxide (in particular SnO₂) and/or a gallium oxide (in particular Ga₂O₃), which are generally deployed in doped form.

In a further preferred embodiment the electrically conducting polymer includes an electrically non-conducting polymer and an electrically conducting additional material, e.g. graphite. However the electrically conducting polymer can also comprise an intrinsically conducting polymer. Preferred intrinsically conducting polymers are polymers with conjugated double bonds, such as polythiophenes and/or polypyrroles for example.

In one alternative embodiment the gas sensors of the odor sensor comprise gas sensors utilizing a mass effect, where mass effect signifies an increase or decrease, in particular an increase in mass. The gas sensors utilizing a mass effect are preferably quartz crystal sensors (QMB/QCM sensors) and/or surface acoustic wave sensors (SAW sensors).

The odor sensor can be based on infrared radiation absorption and to this end can include a transmit element and a receive element, the transmit element infrared radiating laundry items and/or their environment with infrared radiation and the receive element receiving the infrared radiation reflected by the laundry items and/or the walls of the container (generally a drum) and/or the transmitted infrared radiation in the 600 to 4000 cm⁻¹ wave number range and evaluating it for the presence of odorous substances and optionally further volatile, combustible substances. In this process the received infrared radiation in the 600 to 4000 cm⁻¹ range is generally fed to an evaluation circuit. In particular the 1080 to 1300 cm⁻¹ wave number range is used to detect alcohols, ethers, carbon acids and/or esters and the 1350 to 1470 cm⁻¹ and 2850 to 2960 cm⁻¹ wave number ranges are used to detect alkanes. The 1690 to 1760 cm⁻¹ wave number range is preferably used to detect aldehydes, ketones, carbon acids and/or esters.

According to an exemplary embodiment of the invention at least two gas sensors differ in respect of the infrared reaction to different chemical characteristics of a predetermined gas or gas mixture. These characteristics are for example polarity as well as size and shape of the gas molecules. The predetermined gas can be any gas. The gas or gas mixture selected is preferably one which includes an odorous substance of particular interest in the present instance.

According to an exemplary embodiment of the invention it is particularly preferable for the gas sensors to be disposed in the form of at least one sensor array on at least one microchip.

An exemplary embodiment of the inventive laundry treatment appliance preferably includes evaluation means for evaluating the sensor signals received from the sensor signals in respect of the presence of odorous substances. It should be noted here that the human sense of smell is clearly different from the receipt of signals from gas sensors. While the human sense of smell distinguishes between odor-active and odorless gases, gas sensors are characterized by the breadth of the bandwidth of the infrared response to gaseous components. Chemically similar gases are generally detected with similar signal strength.

Different sensor types also vary in the chemical range of the odorous substances to be measures as well as in the infrared measuring technology, said measuring technology being known per se to those skilled in the art.

Since according to an exemplary embodiment of the invention an odor sensor uses a number of gas sensors, which respond differently to different characteristics of the odorous substances (gases) to be determined, the signal strength of each gas sensor will generally be a function of the presence of corresponding molecules of odorous substances, having a specific polarity, molecule size and/or molecule shape. Each gas mixture will therefore generate a characteristic and recognizable signal pattern with the gas sensors. Further signal processing can be clarified in a multidimensional space. The composition of a gas mixture results in a feature space in a signal vector, the direction of which represents the composition and the length of which represents the overall concentration. Based on this measuring principle it is possible to distinguish and recognize gases and gas mixtures. However odor-related qualities such as type of odor and intensity of odor cannot always be detected in this manner. A comparison with olfactory or olfactometric measurements is expedient for this purpose.

In one preferred embodiment of the invention the odor sensor is therefore calibrated using olfactometric measurements. To this end olfactometry according to the European standard EN 13725 is generally used as the standard measuring technology. The odorous substance concentration is measured as an indication of how much an odor specimen has to be diluted before it is odorless to an average person smelling it. It should be noted that the olfactometric measuring method supplies a measurement value, which is subject to a high level of uncertainty, generally between four times and a quarter of a measurement value. For example a measurement value of 1000 OU/m³ (OU: odor units) indicates that a tested specimen has to be diluted to a ratio of 1 to 1000 in order to be odorless for an average person smelling it.

The olfactometric measurements are then compared with sensor signals from the gas sensors and this comparison data is preferably stored as reference signals in a storage unit of a program controller of the laundry treatment appliance.

Alternatively or additionally gas chromatography and/or mass spectroscopy data of specimens of odorous substances, which differ in respect of the type and quantity of odorous substances, can be compared with the sensor signals of the gas sensors. This comparison data is preferably stored as reference signals in a storage unit of a program controller of the laundry treatment appliance.

When evaluating the sensor signals a change in the electrical resistance and/or mass at the individual gas sensors and the ratio of the levels of different sensor signals can be used, these as a whole providing a signal profile for different odorous substances or mixtures of odorous substances.

In one particularly preferred embodiment of the invention the program controller therefore includes a storage unit containing the signal profiles for different odorous substances or mixtures of odorous substances for evaluation purposes.

The laundry treatment appliance of the present invention is particularly a dryer, a washing machine or a washer/dryer.

A dryer generally comprises a process air duct, containing a drying chamber for the items to be dried and generally a heater to heat the process air and a fan to convey the process air.

A dryer is generally operated as a recirculated air or exhaust air dryer, with combined forms also being known. In a recirculated air dryer after condensation of the moisture from damp laundry items in a suitable heat exchanger (air/air heat exchanger, evaporator of a heat pump), moist hot air from the drying chamber is reheated by means of a heater and conducted into the drying chamber. In contrast in an exhaust air dryer an air supply duct and an air discharge duct are present, so that the process air entering the air supply duct from the room passes through the drying chamber and is conducted via the air discharge duct to an air discharge outlet and thus back into the room.

A washing machine generally comprises, in addition to a drum to hold the laundry items to be treated, a tub, a water supply system and a water drainage system with a drainage pump at the base of the tub.

A washer/dryer generally has a combination of the features of a dryer and a washing machine.

A laundry treatment appliance generally has a drum supported in a rotatable fashion, a drive motor for the drum and a heating facility. Switching means are generally also present to rotate and stop the drum.

The odor sensor can be disposed at different points in the laundry treatment appliance.

In a dryer the odor sensor is preferably disposed in the drying chamber and/or in the process air duct behind the drying chamber. If the dryer is a condensation dryer, it is recommended for example that the odor sensor is disposed behind a heat exchanger, in which the moisture in the moist hot process air can be condensed. This minimizes the influence of the moisture on the sensor signal of the odor sensor.

In a washing machine the odor sensor is preferably disposed in the drum. This allows odorous substances to be registered before a washing method is implemented, it being possible then to configure this in respect of the type and quantity of any odorous substances present. When used in a washing machine the odor sensor can be protected optionally by way of a closeable cover from damaging influences due to water or wash liquor before water or wash liquor is introduced.

If an infrared sensor is used as the gas sensor, it should be noted that the transmit element and the receive element together form an infrared sensor. These parts can be disposed in the laundry treatment appliance (particularly in a dryer) a greater or lesser distance apart and can for example be disposed so close to one another that they form an infrared sensor unit. The infrared sensor unit (or its transmit unit and receive unit components) is preferably disposed such that its field of vision covers a maximum surface of the laundry items present in the container. For example the infrared sensor unit can be attached along the axis of rotation of the drum. With such an embodiment the infrared sensor unit can be attached directly to the door of the dryer, through which the laundry items are inserted into the dryer. The sensor can also be attached along other regions of a laundry treatment appliance, allowing a view into the interior of the drum and of the laundry items contained therein. The spectra or wavelengths reflected by a transmitted spectrum of laundry items wet with odorous substances and in some instances volatile, combustible substances allow the nature of said odorous or other substances to be concluded. The same applies to the transmission spectra. The spectra here are either evaluated over a specified spectral range or only at specified frequencies or wave numbers in the infrared range.

The invention also relates to a method for determining the presence of an odorous substance in a laundry treatment appliance with a container to hold laundry items, the laundry treatment appliance having an odor sensor comprising a number of gas sensors, at least two gas sensors being different in respect of their reaction to different chemical characteristics of a predetermined gas or gas mixture, and the gas sensors being used to determine sensor signals as a measure of the presence of odorous substances and said sensor signals being evaluated by comparison with reference signals stored in a program controller of the laundry treatment appliance in respect of the presence of odorous substances.

In one preferred embodiment of this method reference signals are used for the comparison, which were obtained by a combination of gas chromatography and mass spectroscopy measurements and olfactory sensing of a specimen of an odorous substance or a separated mixture of odorous substances.

In an alternative preferred embodiment reference signals are used for the comparison, which were obtained by a combination of olfactometric measurements and the sensor signals.

According to an exemplary embodiment of the invention it is preferable for an acoustic and/or optical signal to be output on detection of a predetermined odorous substance or a predetermined mixture of odorous substances or if a specified threshold value for the concentration of a predetermined odorous substance or a predetermined mixture of odorous substances has been exceeded. According to an exemplary embodiment of the invention it is therefore preferable for the presence of an odorous substance to be displayed by means of a display apparatus on the dryer. This can be done by means of an acoustic and/or optical display apparatus.

The moisture content of the laundry items and/or the moisture content of the process air generally have a significant influence on the measurements of the odor sensor.

It can therefore be proposed that an exemplary embodiment of the inventive method should only be implemented when the moisture content of the process air reaches or drops below a predetermined value.

When an infrared radiation-based odor sensor is used it should in some instances be taken into account that the moisture content of a textile item influences its absorption and/or transmission spectrum in a specified wave number range. Wave number ranges, in which either no such influence is present or the influence on the mutual distinguish ability of laundry items and odorous substances is minor, are preferably selected for measuring the moist laundry items and/or the odorous substances.

Alternatively information which takes into account the dependence of the sensor signals of the odor sensor on the moisture content of the process air or laundry items is stored in a storage unit assigned to the program controller in order to correct the sensor signals accordingly.

An exemplary embodiment of the inventive method allows a laundry treatment program to be tailored to the type and quantity of a determined odorous substance. It can also prevent a laundry treatment program (e.g. a heating program) starting or can terminate an already started laundry treatment program (e.g. a heating program). This is the case in particular if odorous substances indicating a fire are detected.

If odorous substances are detected a laundry treatment method can include the treatment of the laundry items with deodorizing means, for example with a deodorizing composition.

The deodorizing composition is preferably a liquid, i.e. a solution or suspension, and can comprise different treatment substances, such as odor-eliminating substances, fragrances, preferably cyclodextrins and/or microcapsules.

Cyclodextrins are cyclical oligosaccharide compounds with a toroidal structure with a central space, in which apolar organic compounds can be enclosed. This characteristic means they are used today for example in air freshener sprays. The derivatives of a cyclodextrin contained in such products bind the compounds causing unpleasant odors. They also serve as fragrance carriers.

Microcapsules (also referred to as nanocapsules and microspheres) are generally essentially spherical in shape, having an outer and an inner space, generally with a diameter in the range of nanometers up to <1 mm. Substances can be enclosed in this inner space.

The microcapsules have an outer, which is made for example of a film-forming polymer, in which finely dispersed, liquid or solid phases can be enclosed. The outer material can comprise many different types of chemical compounds, e.g. gelatin, gum arabic, agar-agar, lactose, microcrystalline cellulose, modified starch, fatty acid esters, phospholipids, chitosan, alginate and collagen, as well as synthetic polymers such as polyacrylates, polyamides, polyvinyl alcohol or polyvinylpyrrolidone.

The deodorizing composition can be used in solid or liquid form. It is however preferable for it to be applied to the laundry items in liquid form or in the form of fine droplets (spray), for example by spraying, spritzing, flushing or insertion, optionally after a defined program interruption. The deodorizing composition is preferably applied to the laundry items by spraying by means of a suitable introduction apparatus, which is generally provided with a nozzle, while the drum is moved at settling speed or above.

A separate point for this deodorizing step (perhaps “Deodorizing”) can be provided in the menu or on the control panel of the laundry treatment appliance.

A method for ventilating an item of laundry can also be implemented, as described for example in the prior art documents cited here.

The inventive laundry treatment appliance and the inventive method implemented therein have the advantage that odorous substances can be detected in a simple and automatic fashion. It is thus possible to tailor laundry treatment programs to the type and quantity of odorous substances that may be present. Also hazard situations such as a fire or contamination with volatile, combustible substances (e.g. hydrocarbons such as cleaning spirit, alcohols, etc.) can be identified simply and automatically, so that appropriate countermeasures can be instituted quickly. The identification of combustible and harmful substances is independent of the user. There is also greater protection against fire and explosion for loads containing contaminated laundry items.

### BRIEF DESCRIPTION OF THE DRAWINGS

Further details of the invention will emerge from the description which follows of non-restrictive exemplary embodiments of the inventive laundry treatment appliance and the inventive method for its operation, with reference being made to FIGS. 1 and 2. Other embodiments are also possible.

FIG. 1 shows a vertical section through a first embodiment of a laundry treatment appliance in the form of an exhaust air dryer.

FIG. 2 shows a schematic diagram of a vertical section through a second embodiment of a laundry treatment appliance in the form of a washing machine.

### DETAILED DESCRIPTION OF EXEMPLARY EMBODIMENTS OF THE PRESENT INVENTION

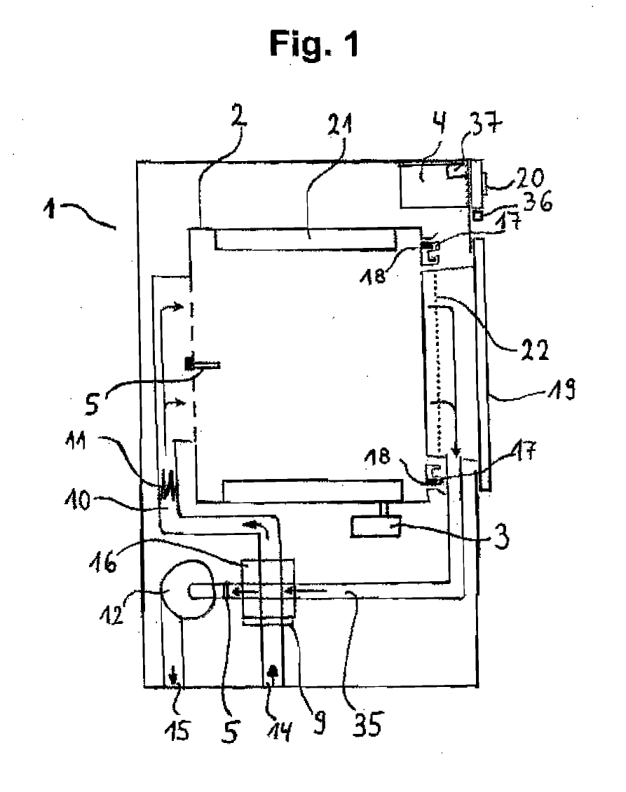

The exhaust air dryer 1 illustrated according to a first embodiment in FIG. 1 has a drum 2 that can be rotated about a horizontal axis as the drying chamber, within which agitators 21 are fixed to move laundry during a drum rotation. Process air is conducted with the aid of a fan 12 from an air supply inlet 14 in an air supply duct 10 by way of a heater 11 through the drum 2 and an air discharge duct 35 to an air discharge outlet 15. With an exhaust air dryer of such embodiment the air supply duct 10, drum 2 and air discharge duct 35 thus form a process air duct. In particular air heated by the heater 11 is directed from behind, i.e. from a side of the drum 2 opposite the door 19, into the drum 2 through the holes in its base, comes into contact there with the laundry to be dried and flows through the loading opening in the drum 2 to a fluff filter 22 within the door 19 sealing the loading opening. The moist hot process air is then deflected downward in the door 19. The process air is fed in the air discharge duct 35 to an air/air heat exchanger 16, in which the hot, moisture-laden process air is cooled and then fed to an air discharge outlet 15. The separated moisture is trapped in a condensate collector 9, from which it can be removed by pumping for example by means of a condensate pump (not shown here).

Room air fed to the dryer 1 by way of the air supply duct 10 is used in the air/air heat exchanger 16 for cooling purposes. This incoming air is heated by the hot, moisture-laden process air and then again by means of the heater 11 before entering the drum 2.

In the embodiment shown in FIG. 1 the drum 2 is supported at the rear of the base by means of a rotary bearing and at the front by means of a bearing plate 17, the drum 2 resting with a rim on a sliding strip 18 on the bearing plate 17 and thus being held at the front end. A motor 3 drives the drum 2. The exhaust air dryer 1 is controlled by way of a program controller 4, which can be controlled by the user by way of a control unit 20. 36 signifies a display means for showing the information contained in a sensor signal measured by an odor sensor 5. 37 signifies an evaluation means for evaluating the sensor signals received from the gas sensors of the odor sensor 5.

In the embodiment shown in FIG. 1 odor sensors 5 are located within the drum 2 and between the fan 12 and the air/air heat exchanger 16.

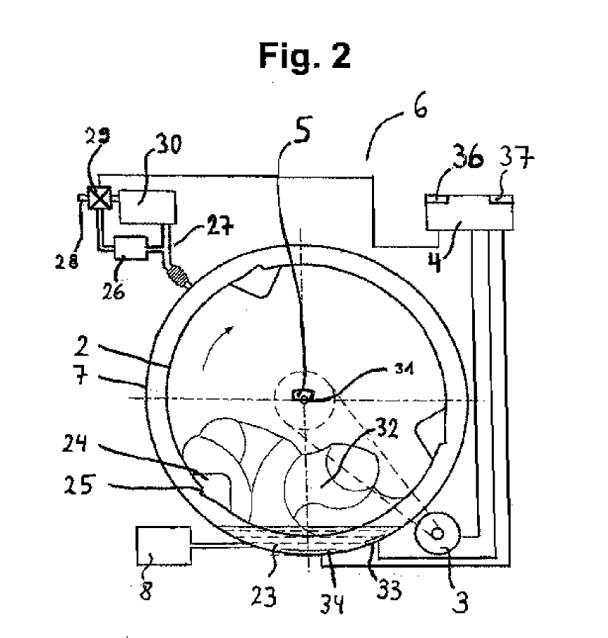

FIG. 2 shows a schematic diagram of a vertical section through a second embodiment in the form of a washing machine.

FIG. 2 is in particular a schematic diagram of the parts of a washing machine 6 that are relevant here, in which washing machine 6 a method described in more detail here can be implemented. The washing machine 6 of the embodiment shown in FIG. 2 has a tub 7, in which a drum 2 is supported in a rotatable fashion and can be operated by a motor 3. In accordance with recent ergonomic findings relating to the operation of such washing machines, the axis of rotation 31 of the drum 2 is directed away from the horizontal by a small angle (e.g. 13°) forward and upward, so that it is easier to access and see into the interior of the drum 2. This arrangement in conjunction with specially shaped laundry agitators 24 and scoops 25 for the wash liquor 23 on the inner surface of the drum shell also intensifies the penetration of wash liquor through the laundry 32.

The washing machine 6 also has a wash liquor supply system, which comprises a water connection fitting for the domestic water supply 28, an electrically controllable valve 29 and a supply line 27 to the tub 7, which passes via a flushable vessel 30, from which the incoming water can transport portions of detergent into the tub 7. A dosing apparatus 26 works in conjunction with the domestic water supply 28 to feed fabric conditioner into the tub 7. A heating facility 34 is also present in the tub 7. The valve 29 and also the heating facility 34 can be controlled by a control facility (“program controller”) 4 as a function of a program sequence, which can be linked to a time schedule and/or the reaching of certain measurement values of parameters such as wash liquor level, wash liquor temperature, speed of rotation of the drum 2, etc., within the washing machine. 33 signifies a sensor for measuring the hydrostatic pressure p in the tub 7. 8 signifies a pump for the liquid present in the tub 7.

With the embodiment in FIG. 2 an odor sensor 5 is disposed in the drum 2, for example on its rear wall, and in the tub 7. 36 signifies a display means for showing the information contained in a sensor signal measured by an odor sensor 5. 37 signifies an evaluation means, which has access to a storage unit of the program controller 4, in which reference signals for different odorous substances and mixtures of odorous substances are stored, which also take into account the influence of moisture on the sensor signals.

When the evaluation means 37 identifies a signal or signal pattern of an odorous substance (sweat; volatile, combustible substances; fire odor), it can influence the further program sequence by way of the program controller 4 and generate for example an optically or acoustically perceptible signal. This can be regulated so that an alarm signal is triggered above a specified concentration.

If there is a threat of danger (for example on detection of odorous substances, which indicate a fire) or if certain predetermined odorous substances occur, the laundry treatment appliance can carry out a program correction independently by not starting or aborting a selected program. It is thus possible to avoid overheating, fire or damage to laundry items due to the action of the odorous substances at high temperatures.

1. A laundry treatment appliance comprising:
a container to hold laundry items; and
an odor sensor comprising at least two gas sensors that are different in respect of their reaction to different chemical characteristics of a predetermined gas or gas mixture.

2. The laundry treatment of claim 1, wherein the laundry treatment appliance is a dryer, a washing machine, or a washer/dryer.

3. The laundry treatment of claim 1, wherein the odor sensor further comprises an electrically conductive material having an electrical resistance that changes on contact with a volatile chemical compound.

4. The laundry treatment appliance of claim 3, wherein the electrically conductive material is a semiconducting metal oxide or an electrically conducting polymer.

5. The laundry treatment appliance of claim 4, wherein the semiconducting metal oxide comprises a stannic oxide and/or a gallium oxide.

6. The laundry treatment appliance of claim 4, wherein the electrically conducting polymer comprises an electrically non-conducting polymer and a conducting additional material.

7. The laundry treatment appliance of claim 4, wherein the electrically conducting polymer comprises an intrinsically conducting polymer.

8. The laundry treatment appliance of claim 1, wherein the gas sensors use a mass effect.

9. The laundry treatment appliance of claim 8, wherein the gas sensors comprise quartz crystal sensors and/or surface acoustic wave sensors.

10. The laundry treatment appliance of claim 1, wherein the gas sensors comprise a sensor array on a microchip.

11. The laundry treatment appliance of claim 1, further comprising evaluation means for evaluating sensor signals received from the gas sensors in respect of the presence of odorous substances.

12. A method for determining the presence of an odorous substance in a laundry treatment appliance with a container to hold laundry items, and an odor sensor with at least two gas sensors that are different in respect of their reaction to different chemical characteristics of a predetermined gas or gas mixture, the method comprising:
determining sensor signals based upon a measure of the presence of odorous substances with the gas sensors;
comparing the sensor signals with reference signals in a program controller.

13. The method of claim 12, further comprising obtaining the reference signals by a combination of gas chromatography and mass spectroscopy measurements and olfactory sensing of a specimen of an odorous substance or a separated mixture of odorous substances.

14. The method of claim 12, further comprising obtaining reference signals by a combination of olfactometric measurements and the sensor signals.

15. The method of claim 12, further comprising displaying a presence of an odorous substance on a display of the laundry treatment appliance.
